(19) United States
(12) Patent Application Publication
(10) Pub. No.: US 2020/0251211 A1
(43) Pub. Date: Aug. 6, 2020

(54) Mixed-Reality Autism Spectrum Disorder Therapy

(71) Applicant: Mississippi Children's Home Services, Inc. dba Canopy Children's Solutions, Jackson, MS (US)

(72) Inventors: Shauna McKinney, Winter Garden, FL (US); Terry Hight, Flowood, MS (US); John Damon, Madison, MS (US); Jim Moore, Purvis, MS (US)

(21) Appl. No.: 16/781,423

(22) Filed: Feb. 4, 2020

Related U.S. Application Data
(60) Provisional application No. 62/800,910, filed on Feb. 4, 2019.

(51) Int. Cl. G16H 40/67; G16H 20/70; G06F 3/0484; G06F 3/01; G06K 9/00; G09G 5/37; A61M 21/00

(52) U.S. Cl. CPC G16H 40/67; A61M 2205/3592; G06F 3/0484; G06F 3/013; G06K 9/00302; G09G 5/37; A61M 21/00; G09G 2354/00; A61M 2021/005; A61M 2205/3306; A61M 2205/3375; A61M 2230/63; A61M 2205/502; A61M 2209/088; A61M 2205/3584; G16H 20/70

(57) ABSTRACT

The present disclosure relates to a mixed-reality therapy system is configured to provide tasks and prompts to a user and monitor the user's responses.

### CROSS REFERENCE TO RELATED APPLICATIONS

This application claims priority to and the benefit of pending U.S. Provisional Application No. 62/800,910 filed Feb. 4, 2019.

### FIELD OF THE DISCLOSURE

The present disclosure pertains to the field of Autism Spectrum Disorder treatment. More specifically, the present disclosure pertains to a system for assessing and delivering mixed-reality therapies to patients with Autism Spectrum Disorder.

### BACKGROUND

Therapies for Autism Spectrum Disorder patients can be difficult to administer. Information about patient performance and responses to treatment programs is difficult to record precisely, and can be interpreted differently by different treatment providers. In addition, it can be difficult to administer treatment methodologies consistently across different patients or even the same patient over time or when different treatment providers are involved. It is also difficult to control perception of a patient during the treatment process or to record the patient's sensory perceptions. Improved techniques for treating Autism Spectrum Disorder are generally desirable.

### BRIEF DESCRIPTION OF THE DRAWINGS

To further illustrate the advantages and features of the present disclosure, a more particular description of the invention will be rendered by reference to specific embodiments thereof which are illustrated in the appended drawings. It is appreciated that these drawings are not to be considered limiting in scope. The invention will be described and explained with additional specificity and detail through the use of the accompanying drawings in which:

FIG. 1 depicts a mixed-reality therapy system in accordance with some embodiments of the present disclosure.

FIG. 2 depicts a wearable device of a mixed-reality therapy system in accordance with some embodiments of the present disclosure.

FIG. 3 depicts a wearable device display of a mixed-reality therapy system in accordance with some embodiments of the present disclosure.

FIG. 4 depicts a treatment provider terminal of a mixed-reality therapy system in accordance with some embodiments of the present disclosure.

FIG. 5 depicts a server of a mixed-reality therapy system in accordance with some embodiments of the present disclosure.

FIG. 6 is a flowchart depicting an exemplary method for delivering therapy with a mixed-reality therapy system in accordance with some embodiments of the present disclosure.

### DETAILED DESCRIPTION

A mixed-reality therapy system is configured to provide tasks and prompts to a user and monitor the user's responses as part of providing therapy or treatment to the patient. This is opposed to other approaches that may only use devices which collect data to perform an evaluation of a patient. In some embodiments, the mixed-reality therapy system is configured to teach individuals with Autism to “learn how to learn,” enabling them to develop in important ways, such as by acquiring life-long skills. The system can accomplish this using techniques such as sentiment analysis. The system can be used to provide treatment in the home, school, office or any other setting. Further, the system can be configured to provide treatment via Applied Behavioral Analysis (ABA) protocols at a self-paced progression.

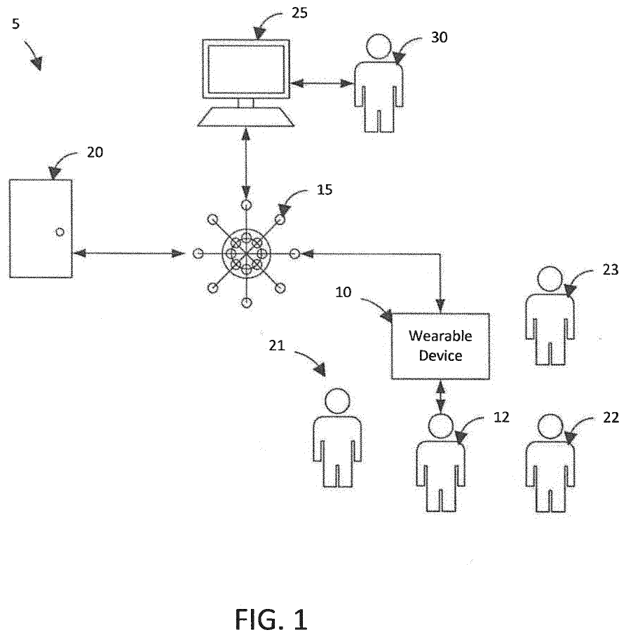

As shown in FIG. 1, in some embodiments, a mixed-reality, evidence-based Autism therapy system 5 can include a wearable device 10 configured to provide a mixed-reality experience when worn by a user 12. In an embodiment, the wearable device 10 may be configured to display graphical objects to the user 12 that are indicative of information such as tasks and prompts that the user 12 can perceive and act upon. The graphical objects displayed by wearable device 10 also can include objects such as customizable avatars that are configured to affect a perception of others (e.g., people 21, 22, and 23) developed by the user 12.

The system 5 also may include a network 15, server 20 and treatment provider terminal 25 used by a treatment provider 30. Each of the wearable device 10, server 20 and treatment provider terminal 25 may be configured to communicate with one another via the network 15 and the network 15 itself. The system 5 can include various other components and perform other functionality consistent with the present disclosure in other embodiments.

In some embodiments, the network 15 can be various types of networks, such as a wide area network (WAN), local area network (LAN) LAN, or other network. A single network 15 is shown in FIG. 1, but in some embodiments, network 15 can comprise various quantities of networks. In an embodiment, the network 15 may be configured to communicate via various protocols (e.g., TCP/IP, Bluetooth, WiFi, etc.), and can comprise either or both a wireless network, wired network, or various combinations thereof.

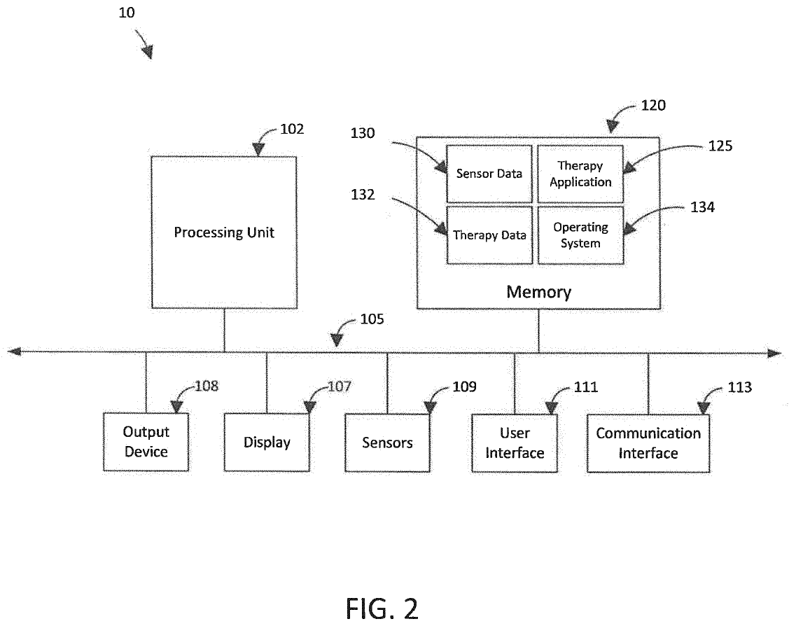

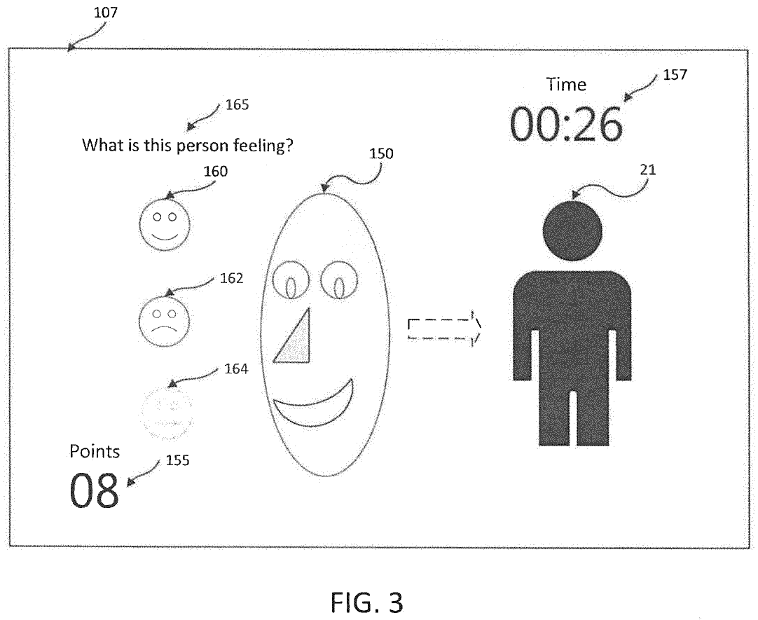

FIG. 2 shows an exemplary embodiment of a wearable device 10. The wearable device 10 may be various devices, but in some embodiments, the device 10 is a pair of mixed-reality smartglasses such as a Microsoft® HoloLens™ or similar device. The wearable device 10 can be a head-mounted device, and can include a display 107 configured to display graphical objects (e.g., sentimental object, emoji objects 160, 162, and 164 of FIG. 3) to the user 12. The device can include a processing unit 102 that is configured to execute instructions stored in memory 120. The processing unit 102 can be implemented in hardware and configured to communicate with and drive the other resources of the device 10 via internal interface 105, which can include one or more buses.

Display 107 can be an interactive display that is configured to display graphics and graphical objects to the user 12. The display 107 can implement a graphical user interface (GUI) and can have variable transparency controlled by the processing unit 102 (e.g., executing instructions stored in memory 120). The display 107 can be configured to implement an application such as therapy application 125 running on operating system 134, each of which is implemented in software and stored in memory 120. Therapy application 125 can generate graphics, such as graphical object 150 and emoji objects 160, 162 and 164 of FIG. 3, and display the graphics for the user 12 via display 107. The display 107 thus can be configured to allow a user 12 to see and perceive the user's environment (e.g., objects, family, friends, treatment providers, etc.) alongside the graphics. In some embodiments, the display 107 can be a touch screen configured to receive touch inputs or optical inputs based on the user's 12 eye position and associate them with graphical objects.

Returning to FIG. 2, the device 10 can include an output device 108 configured to provide an output such as sound to the user 12, such as one or more speakers. The output device 108 can be one or more devices, such as a pair of earphones.

Sensors 109 can include one or more various types and quantities of sensors in order to detect data indicative of various aspects of the environment and store the data in sensor data 130. Sensors 109 include light sensors (e.g., optical scanners, infrared, etc.), sound sensors (e.g., microphones, acoustic receivers, etc.), touch sensors (e.g., pressure-sensitive surfaces, etc.) or other sensor types. The sensors 109 can be configured as passive or active sensors. The sensors 109 can be configured to track facial movements of the user 12, such as eye movement and changes in positions of facial features of the user 12. In some embodiments, one or more of the sensors 109 can be configured to sense data such as inputs of the user 12, such as by scanning one or more eyes of a user 12, receiving verbal inputs from the user 12, or receiving tactile inputs from the user 12. Such inputs also can be provided to user interface 111, which can be various devices configured to receive inputs from user 12 such as a microphone, keyboard, mouse or other device (not specifically shown in FIG. 2).

Communication interface 113 can include various hardware configured to communicate data with a network or other devices (e.g., other devices 10, the network 15, server 20, treatment provider terminal 25, etc.). The interface 113 can communicate via wireless or wired communication protocols, such as radio frequency (RF) or other communication protocols.

Therapy data 132 can include information based on progress of the user's 12 most recent use of the therapy application 125 or information from one or more of the user's 12 treatment sessions with a caregiver. Therapy data 132 can be indicative of data received or provided by the therapy application 125 during use, including data sensed by sensors 109 and data received via user interface 111, communication interface 113, or otherwise.

In some embodiments, therapy data 132 can include any suitable information delivered or collected by the device 10 during use of the therapy application 125, including responses provided by the user 12 and data sensed by sensors 109 (e.g., eye movements, verbal responses, facial expressions, field of view of the user 12 during use, data displayed via display device 107, etc.). The therapy data 132 also can include data indicative of data displayed to the user during use via the display 107 and data indicative the environment that is visible to the user 12 (e.g., recordings of video, audio, eye movement, or other data sensed by sensors 109 and stored in sensor data 130). Thus, the therapy data 132 can include data indicating information available to the user 12 while wearing the device 10 and during use and the user's response to such information. In this regard, therapy data 132 may include suitable information for evaluating performance of a user 12 and allow assessment of the user's 12 skill level and progress for modification of future therapy delivered to the user 12 (either by the therapy application 125 or a treatment provider). The data 132 can also include information from analysis of treatment provided to the user (e.g., user response and performance).

The therapy data 132 also can include information about the user 12, including information needed to select an appropriate module or exercise for the user 12 to experience when using therapy application 125. Exemplary data can include a gender, age, identity, and indicators of the user's 12 performance history, skill levels, and other information associated with ABA treatment methodology.

Therapy application 125 can include instructions configured to assess skill level of user 12 and implement and provide a mixed-reality therapy regimen to the user 12 via wearable device 10. In an embodiment, the features of therapy application 125 can be selected and structured based on ABA methodology. The therapy application 125 can be configured to provide treatment at essentially any location where the user 12 can use the wearable device 10, such as in the user's home, school, a treatment provider's facility or otherwise.

The therapy application 125 can use information in therapy data 132 and sensor data 109 to generate and provide content specifically selected for the user 12. Therapy application 125 can include various instructions and algorithms configured to use information about treatment status of the user 12 to adjust content provided to the user 12 during use. For example, the therapy application 125 can use information from therapy data 132 to perform an estimation of the user's progress through a treatment regimen associated with the user 12 either using therapy application 125 or via sessions with a treatment provider and modify content of a module or lesson (e.g., tasks, prompts, rewards, etc.). The application 125 can use information from sensor data 109 indicative of the user's eye movements, facial expressions, or verbal responses to modify a module or lesson (e.g., dimming graphics provided via display 107 if a user response indicates that the user 12 is overstimulated). In some embodiments, the therapy application 125 can modify and improve content provided to the user 12 during use by applying one or more artificial intelligence (“AI”) or machine learning algorithms to one or more of therapy data 132 or sensor data 109. Other features of therapy application 125 may be present in other embodiments.

In some embodiments, the therapy application 125 can have modules and exercises designed to treat Autism Spectrum Disorder using ABA methodologies, although other types of methodologies and treatment regimens are possible. In some embodiments, the therapy application 125 can provide graphics indicative of tasks, such as questions, prompts, milestones, achievements, rewards and other aspects of the therapy application 125. The therapy application 125 can be implemented as a game played by the user 12, where progress through the game corresponds to progress of the user 12 through a program using ABA methodology. The therapy application 125 can be configured to recognize and reward achievements of the user 12 during use, such as via affirmative messaging or otherwise.

In an exemplary operation of the therapy application 125, a module can begin when the user 12 begins wearing the device 10 or provides an input indicating the module should begin. As shown in FIG. 3, a sentimental graphical object 150 associated with a preference of the user 12 (e.g., a favorite cartoon character, animal, or other object) can be displayed via display 107 and overlaid on another person (e.g., people 21, 22, 23) to encourage and enhance social interaction between the user 12 and the person. The therapy application 125 can display a point total 155 reflecting an amount of points the user 12 has achieved for the module. The application 125 can display a timer 157 indicating one or more amounts of time that have elapsed (e.g., since the module began, since a task began, etc.). The timer 157 also can be a countdown timer. The application 125 can modify the point total 155 and timer 157 values based on progression of the module and inputs, such as from the user 12 or treatment provider 30.

In some embodiments, a “sentiment” task may be provided by the application 125, including a textual prompt 165 that instructs the user 12 to “go find someone.” The application 125 may monitor information from sensor data 130 and determine when the user 12 is looking at a person (e.g., person 21). The application 125 may determine a position of the person 21 detected in sensor data 130 and identify a plurality of pixels of the display 107 associated with a position of all or a portion of the person 21. Referring to FIG. 3, the application 125 may generate and overlay the sentimental graphical object 150 over one or more of the plurality of pixels of the display 107 such that the user 12 sees the graphical object 150 instead of the person 21. The application 125 may be configured to detect emotions, physical movements and facial expressions of the person 21 using sensor data 130 and to control the graphical object 150 to mimic the emotions, movements and facial expressions of the person 21.

Thereafter, the user 12 may be prompted by the prompt 165 to “say hello.” The application 125 can display a prompt 165 asking “what is this person feeling?” as well as a plurality of graphical emoji objects depicting various different emotional states (e.g., a smile, frown, surprise, etc.). The application 125 may then receive an input from the user 12 indicative of a selection of the user 12 of a graphical emoji object 160, 162, 164 associated with the user's perception of an emotional state of the person 21. Object 160 of FIG. 3 indicates a happy emotional state, object 162 indicates a sad emotional state, and object 164 indicates a neutral emotional state, but other emotional states can be indicated by other graphical emoji objects in some embodiments.

The application 125 can determine whether a selected emoji 160-164 is associated with a state that matches a detected emotional state of the person 21. If so, the application 125 can determine that the user 12 has answered correctly and award the user 21 points that can be reflected in point total 155. The application 125 can also display a celebratory character for the user 12 via display 107 (not specifically shown).

If the application 125 determines that the user 12 has answered incorrectly, the application 125 can decrement the number of graphical emoji objects displayed to the user 12 as available selections and ask the user “what is this person feeling?” again. As an example, emoji object 164 may be removed as an available option (e.g., greyed out or removed from the display 107) by the application 125 following an incorrect response from the user 12. The application 125 may continue to decrement the number of graphical emoji objects 160-164 displayed as available options until the user 12 selects the correct answer or a time limit is reached (e.g., time on timer 157 expires).

The application 125 can provide an additional prompt to the user 12 if the user 12 answers a question from prompt 165 or completes a task correctly. Displayed tasks or prompts can increase in complexity if desired when the user 12 answers a question or completes a task correctly, or achieves a certain score. Reward indicators (e.g., achievement and congratulatory graphics) can also modified to reflect increased task or question complexity. In some embodiments, the application 125 may control a transparency of the graphical object 150, such as based on progress of the user 12 within the sentiment task. In this regard, increase in transparency of the object 150 can permit the user 12 to perceive more of the person 21 and less of the graphical object 150 based on whether the user 12 is correctly completing tasks or answering questions.

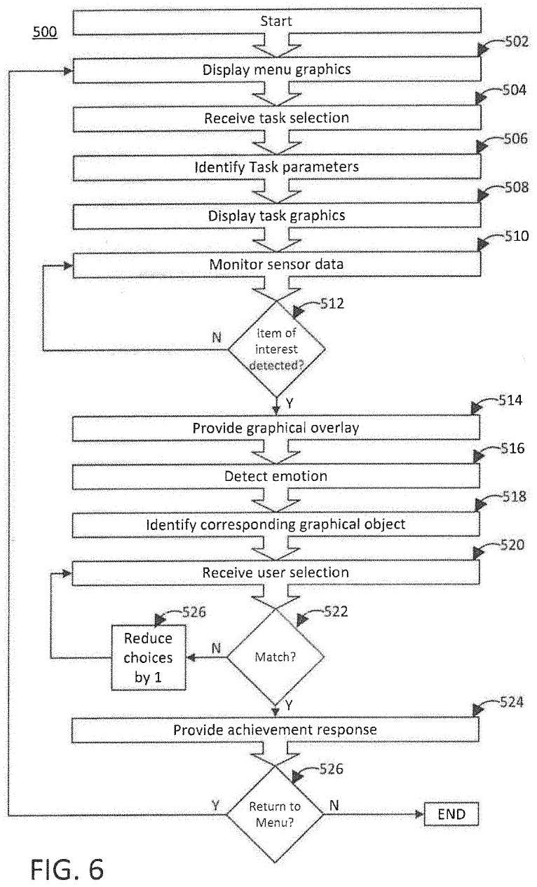

Additional description of an exemplary operation of the therapy application 125 is discussed in more detail below with regard to FIG. 6.

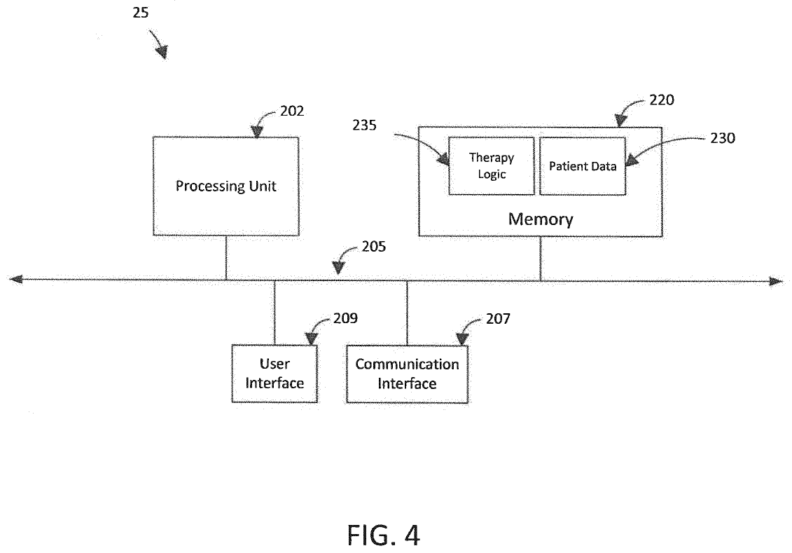

FIG. 4 shows an exemplary embodiment of a treatment provider terminal 25 for use by a treatment provider 30 (e.g., an ABA treatment provider). Terminal 25 can be various devices, including a desktop computer or smartphone such as an iPhone®, Android® or other device. The terminal 25 can include a processing unit 202 that is configured to execute instructions stored in memory 220, such as therapy logic 235. The processing unit 202 can be implemented in hardware and configured to communicate with and drive the other resources of the terminal 25 via internal interface 205, which can include one or more buses.

Communication interface 207 can include various hardware configured to communicate data with a network or other devices (e.g., devices 10, the network 15, server 20, other treatment provider terminal 25, etc.). The interface 207 can communicate via wireless or wired communication protocols, such as radio frequency (RF), Bluetooth, or other communication protocols.

User interface 209 can be configured to receive inputs and provide outputs to a user such as treatment provider 30. The interface 209 can be implemented as a touchscreen in some embodiments, but also can be one or more devices such as a keyboard, mouse or other device in some embodiments.

Patient data 230 is implemented in software and stored in memory 220, and can include information about one or more user 12 associated with one or more accounts serviced by the server 20 (e.g., accounts of one or more treatment providers, schools, etc.) and can include information needed to select an appropriate module or exercise for the user 12 to experience when using therapy application 125. Exemplary data can further information about a user's 12 performance history, skill levels, therapy progress, medical history, or other information suitable for assessment and treatment of a user for which modification of the therapy application 125 may be desirable. The patient data 230 also can include data (e.g., sensor data 130 and therapy data 132) uploaded from one or more devices 10, such as performance data of a user 12 while using therapy application 125 and any interaction by a treatment provider 30 with one or more users 12 via one or more devices 10.

Therapy logic 220 is implemented in software and can be configured to allow a treatment provider 30 to control, monitor, assess, and modify mixed-reality therapy provided to one or more users 12 via therapy application 125 running on respective devices 10. The logic 220 can use data from patient data 230 to generate an output for the treatment provider 30 indicative of performance of a user 10 while using therapy application 125. The logic 220 can receive inputs from the treatment provider 30 indicative of modifications or other information related to therapy application 125 and store the inputs in patient data 230. In an embodiment, the logic 220 can be configured to permit the treatment provider 30 to receive information about and control operation of therapy application 125 running on one or more devices 10 of one or more users 12 essentially in real-time. In some embodiments the logic 220 can communicate information from patient data 230 to one or more servers 20, such as via network 15.

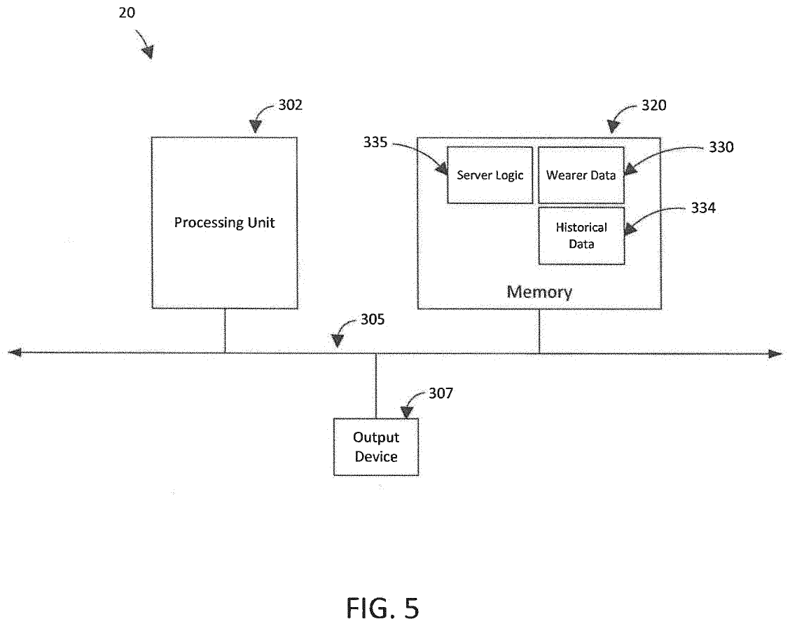

FIG. 5 shows an exemplary embodiment of a server 20. Server 20 can include a processing unit 302 that is configured to execute instructions stored in memory 320, such as server logic 335. The processing unit 302 can be implemented in hardware and configured to communicate with and drive the other resources of the server 20 via internal interface 305, which can include one or more buses. A data interface 307 can include various hardware configured to communicate data with a network (e.g., network 15) or other devices (e.g., other devices 10, the network 15, treatment provider terminal 25, etc.).

The application data 330 is implemented in software and stored in memory 320. The data 330 can include information from one or more devices 10 about performance of therapy application 125. Historical data 334 is implemented in software and stored in memory 320. The data 334 can include information stored as patient data 230 at a plurality of treatment provider terminals 25. In some embodiments, historical data 334 also can include similar information that is available for patients with Autism Spectrum Disorder globally.

Server logic 335 can be implemented in software and stored in memory 320. The logic 335 can use information in application data 330 and historical data 334 to generate updates for therapy application 125 and provide the updates to devices 10 serviced by the server. The server logic 335 can include artificial intelligence or machine learning algorithms, and can apply such algorithms to the data stored in memory 320 to modify instructions or functionality of therapy application 125. Such modifications can be implemented in an update for the therapy application 125, which can be communicated to one or more devices 10 via network 15 and installed at the one or more devices 10. Server logic 335 may be configured to use such modifications for other purposes, such as modification or design of education studies regarding Autism Spectrum Disorder, improvement of treatment provider or treatment provider training and development, or for provision to other users (e.g., via network 15) for various purposes.

An exemplary method 500 for delivering mixed-reality therapies is shown in FIG. 6. At step 502, application 125 can display menu graphics with task selections. At step 504, the application 125 can receive a task selection for the user 12 for the task “go find someone.” At step 506, the application 125 can identify parameters for the task (e.g., sentiment or other task) and at step 508, may display the task graphics via display 107. The graphics can include a prompt to “go find someone.” The application may monitor sensor data and display pixels at step 510.

If an item of interest for the particular task “go find someone” (e.g., person) is not detected at step 512, processing may return to step 510 and monitoring may continue until such an item is detected. If an item of interest is detected, at step 512, processing may continue to step 514 and a graphical overlay may be provided with a sentimental graphical object and one or more graphical emoji objects. At 516, the application 125 may detect emotion of the person based on sensor data 130 and may control the sentimental graphical object to mimic the person. At step 518, the application may identify a correct graphical emoji object from the plurality of objects associated with the person's emotions and wait for a user selection.

At step 520, the user may select a graphical emoji object. At step 522, the application 125 may receive the selection and determine whether the selected object matches the object associated with the person's emotions. If so, the application 125 can provide an achievement response at step 524, which can include celebratory messaging, points increments or otherwise. If the selection does not match, the application 125 may decrement a number of available emoji object choices by 1 and return to step 520 to allow the user to select again.

After the application has provided an achievement response at step 524, the application 125 may determine at step 526 whether additional tasks should be provided or whether to return to the application menu. If the application should return to the menu, processing may return to step 502. If not, processing may end.

Although particular embodiments of the present disclosure have been described, it is not intended that such references be construed as limitations upon the scope of this disclosure except as set forth in the claims.

What is claimed is:

1. A therapy system comprising:
a. a wearable device, wherein the wearable device comprises a processing unit configured to display graphical objects to the person wearing the wearable device;
b. a treatment provider terminal, wherein the wearable device and treatment provider terminal are in communication with one another over a network; and
c. one or more sensors configured to track facial movements of the person wearing the wearable device.

2. The system of claim 1 wherein the one or more sensors are selected from the group consisting of light sensors, sound sensors or touch sensors.

3. The system of claim 1 wherein the graphical objects are emoji objects or sentimental graphical objects.

4. The system of claim 1 further comprising an user interface which receives inputs from the person wearing the wearable device.

5. The system of claim 2 wherein at least one of the one or more sensors track the person wearing the wearable device's eye movements or facial expressions.

6. The system of claim 1 wherein the treatment provider terminal provides instruction to the wearable device concerning which graphical object to display.

7. The system of claim 1 wherein the wearable device is smart glasses.

8. The system of claim 5 wherein the wearable device is smart glasses.

9. A therapy system comprising:
a. a wearable device, wherein the wearable device comprises a processing unit configured to display graphical objects to the person wearing the wearable device;
b. a treatment provider terminal, wherein the wearable device and treatment provider terminal are in communication with one another over a network, wherein the treatment provider terminal provides instruction to the wearable device concerning which graphical object to display;
c. one or more sensors configured to track facial movements of the person wearing the wearable device, wherein the one or more sensors are selected from the group consisting of light sensors, sound sensors or touch sensors; and
d. an user interface which receives inputs from the person wearing the wearable device.

10. The system of claim 7 wherein at least one of the one or more sensors track the person wearing the wearable device's eye movements or facial expressions.

11. The system of claim 9 wherein the wearable device is smart glasses.

12. The system of claim 10 wherein the wearable device is smart glasses.
